(19) United States
(12) Patent Application Publication
(10) Pub. No.: US 2020/0146668 A1
(43) Pub. Date: May 14, 2020

(54) BONE ANCHOR

(71) Applicant: Helix Fixation, Inc., Pleasanton, CA (US)

(72) Inventor: John F. KRUMME, Bainbridge Island, WA (US)

(21) Appl. No.: 16/678,843

(22) Filed: Nov. 8, 2019

Related U.S. Application Data
(60) Provisional application No. 62/757,567, filed on Nov. 8, 2018.

(51) Int. Cl. A61B 17/04

(52) U.S. Cl. CPC A61B 17/0401; A61B 2017/00867

(57) ABSTRACT

Bone implant systems and methods of using them, including bone implants, bone anchors, precursors for bone anchors, methods for preparing bone anchors, methods for preparing bone anchor precursors, assemblies for inserting bone anchors, methods for preparing such assemblies, methods for using such assemblies to insert bone anchors, components for use in such assemblies, and substrates having the bone anchors secured therein. The bone implants may include a monolithic article made from a shape memory alloy.

### CROSS REFERENCE TO RELATED APPLICATIONS

This application claims priority to U.S. Provisional Patent Application No. 62/757,567 filed Nov. 8, 2018 and title “Bone Anchor” which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This disclosure relates to bone implant systems and methods of using them, including bone implants, bone anchors, precursors for bone anchors, methods for preparing bone anchors, methods for preparing bone anchor precursors, assemblies for inserting bone anchors, methods for preparing such assemblies, methods for using such assemblies to insert bone anchors, components for use in such assemblies, and substrates having the bone anchors secured therein.

### BACKGROUND

Bone anchors are used extensively in open and minimally invasive surgery to reattach tissue to bone. The bone anchor is secured to the bone and one or more sutures attached to the bone anchor are used to secure the tissue to the bone. The tissue can be soft tissue anywhere in the body, e.g. a torn rotator cuff in a shoulder or a torn ligament. One type of bone anchor is disclosed in U.S. Pat. Nos. 8,721,650 and 9,539,001. The entire disclosure of each of those patents is incorporated herein by reference for all purposes. With over 3 million cases of rotator cuff tears and over 200 anterior cruciate ligament tears in the knee every year in the United States alone, there is a need for improved bone anchor devices, systems, and methods that are cost-effective, easy to use, and improve recovery. Provided herein are bone anchor devices, systems, and methods that are cost-effective, easy to use, and improve recovery.

### SUMMARY OF THE DISCLOSURE

The present disclosure relates to bone implant systems and methods of using them, including bone implants, bone anchors, precursors for bone anchors, methods for preparing bone anchors, methods for preparing bone anchor precursors, assemblies for inserting bone anchors, methods for preparing such assemblies, methods for using such assemblies to insert bone anchors, components for use in such assemblies, and substrates having the bone anchors secured therein.

One aspect of the disclosure provides a precursor for a bone anchor having a monolithic article. In some embodiments, the precursor includes a body portion including a first planar body surface, a second planar body surface which is parallel to the first planar body surface, and a peripheral body surface joining the first and second body surfaces. In some embodiments, the precursor includes at least two anchor arms which are separated by a slot and which extend away from the body portion, each of the anchor arms including (a) a first planar arm surface which is coplanar with the first planar body surface, (b) a second planar arm surface which is parallel to the first planar arm surface and parallel to the second planar body surface, (c) a peripheral inner arm surface which is adjacent the slot and which joins the first and second arm surfaces, and (d) a peripheral outer arm surface which is remote from the slot and which joins the first and second arm surfaces. In some embodiments, the monolithic article includes a shape memory material.

In some embodiments, the monolithic article includes a shape memory material that includes a nickel-titanium alloy. In some embodiments, the monolithic article includes a shape memory material including a nickel-titanium alloy containing about 55-56% by weight of nickel. In some embodiments, the monolithic article includes a shape memory material including a nickel-titanium alloy containing about 44-45% by weight of titanium.

In some embodiments, body portion includes an orifice configured to pass a suture therethrough. Some such embodiments further include a slidable cinch bar in the orifice.

In some embodiments, the precursor includes no more than two anchor arms. In some embodiments, the precursor includes four anchor arms.

Another aspect of the disclosure provides a bone anchor having a monolithic article.

In some embodiments, the precursor includes a body portion including a first planar body surface, a second planar body surface which is parallel to the first planar body surface, and a peripheral body surface joining the first and second body surfaces. In some embodiments, the precursor includes at least two arms which are separated by a slot and which extend away from the body portion in opposite directions, each of the arms comprising (a) a first arm surface, (b) a second arm surface which is parallel to the first arm surface, (c) a peripheral inner arm surface which is adjacent the slot and which joins the first and second arm surfaces, and (d) a peripheral outer arm surface which is remote from the slot and which joins the first and second arm surfaces. In some embodiments, the monolithic article includes a shape memory material.

In some embodiments, the shape memory material includes a nickel-titanium alloy. In some embodiments, the shape memory material includes a nickel titanium alloy containing about 55-56% by weight of nickel. In some embodiments, the shape memory material includes a nickel titanium alloy containing about 44-45% by weight of titanium.

In some embodiments, the body portion includes an orifice configured to pass a suture therethrough. Some such embodiments further include a slidable cinch bar in the orifice.

In some embodiments, the precursor includes no more than two anchor arms. In some embodiments, the precursor includes four anchor arms.

Another aspect of the disclosure provides a device for delivering a bone anchor. Some embodiments of the device including (1) a proximal handle; and (2) a hollow distal section attached to the handle and having an exposed distal end. In some embodiments, the exposed distal end is configured to receive a bone anchor including a body portion having a first planar body surface, a second planar body surface which is parallel to the first planar body surface, and a peripheral body surface joining the first and second body surfaces. Some embodiments of the bone anchor include at least two anchor arms which are separated by a slot and which extend away from the body portion in opposite directions. In some embodiments, each of the arms include (a) a first arm surface, (b) a second arm surface which is parallel to the first arm surface, (c) a peripheral inner arm surface which is adjacent the slot and which joins the first and second arm surfaces, and (d) a peripheral outer arm surface which is remote from the slot and which joins the first and second arm surfaces. In some embodiments, the anchor arms are generally coplanar with the body portion.

Some embodiments of the bone anchor delivery device further include an element configured to force the bone anchor out of the exposed distal end after the device has been placed over a hole in a bone.

Some embodiments of the bone anchor delivery device further include a pushrod configured to pass through the handle and/or the hollow distal section and to apply a force on a bone anchor in the exposed distal end.

Some embodiments of the bone anchor delivery device further include a knob or handle on a proximal end of the pushrod wherein pushing on the knob or handle pushes the pushrod through the hollow distal section and applies a force on the bone anchor in the exposed distal end. Some embodiments of the bone anchor delivery device further include a knob or handle on a proximal end of the pushrod wherein rotating or turning the knob or handle pushes the pushrod through the hollow distal section and applies a force on the bone anchor in the exposed distal end. In some embodiments of the bone anchor delivery device, a distal end of the pushrod is configured to connect with a proximal end of the bone anchor. In some embodiments of the bone anchor delivery device, a distal end of the pushrod is configured to fit inside the slot.

Another aspect of the disclosure provides a method of delivering a bone anchor with a delivery device. Some embodiments include the step of obtaining a bone anchor having a body portion having a first planar body surface, a second planar body surface which is parallel to the first planar body surface, and a peripheral body surface joining the first and second body surfaces and at least two shape memory cooled arms which are separated by a slot and which extend away from the body portion in opposite directions, each of the arms including (a) a first arm surface, (b) a second arm surface which is parallel to the first arm surface, (c) a peripheral inner arm surface which is adjacent the slot and which joins the first and second arm surfaces, and (d) a peripheral outer arm surface which is remote from the slot and which joins the first and second arm surfaces.

Some embodiments include the step of moving the cooled anchor arms of the bone anchor towards a substantially parallel configuration. Some embodiments include the step of inserting the anchor arms with the substantially parallel configuration into the hollow end section of a bone anchor delivery device having a handle and a hollow end section attached to the handle and which has a distal end.

Yet another aspect of the disclosure provides a method of making a bone anchor precursor. Some embodiments include the step of modifying a preformed ribbon of a shape memory alloy to create a bone anchor feature. Some embodiments include the step of cutting the ribbon into a plurality of discrete parts. In some embodiments, the preformed ribbon is the same width as the widest width of the bone anchor.

In some embodiments, the created bone anchor feature includes at least one of: an anchor arm, an orifice, a slot between two anchor arms, and a slot continuous with the orifice. In some embodiments, the created bone anchor feature includes at least two anchor arms, an orifice, and a slot.

In some embodiments, the shape memory material includes a nickel-titanium alloy. In some embodiments, the shape memory material includes a nickel titanium alloy containing about 55-56% by weight of nickel. In some embodiments, the shape memory material includes a nickel-titanium alloy containing about 44-45% by weight of titanium.

In some embodiments, each of the plurality of discrete parts includes a bone anchor precursor.

### BRIEF DESCRIPTION OF THE DRAWINGS

The novel features of the invention are set forth with particularity in the claims that follow. A better understanding of the features and advantages of the present disclosure will be obtained by reference to the following detailed description that sets forth illustrative embodiments, in which the principles of the disclosure are utilized, and the accompanying drawings of which:

FIG. 1, FIG. 2, and FIG. 3 are top, side and perspective views of a precursor for a bone anchor which has two anchor arms.

FIG. 4 is a perspective view of a bone anchor with two anchor arms, obtained by deforming and heat setting the anchor arms of the bone anchor precursor shown in FIG. 1-FIG. 3, so that the anchor arms extend from the body portion in opposite directions,

FIG. 5, FIG. 6, and FIG. 7 are top, side, and perspective views of a precursor for a bone anchor which has four anchor arms.

FIG. 8 is a perspective view of a bone anchor obtained by deforming and heat setting the anchor arms of the bone anchor precursor shown in FIG. 5-FIG. 7, so that the anchor arms extend from the body portion in opposite directions.

FIG. 9, FIG. 10 and FIG. 11 are top, side and perspective views of a precursor for a bone anchor which has four anchor arms and which has an orifice into which is fitted a cinch bar.

FIG. 12 is a perspective view of a bone anchor obtained by deforming and heat setting the anchor arms of the bone anchor precursor shown in FIG. 9-FIG. 11 so that the anchor arms extend from the body portion in opposite directions.

FIG. 13A is a side view of a bone anchor delivery device with a bone anchor in place for delivery to a bone.

FIG. 13B is a perspective view of the bone anchor delivery device shown in FIG. 13A.

FIG. 13C is a perspective view of the bone anchor delivery device shown in FIG. 13A and FIG. 13B without a bone anchor.

FIG. 13D is a perspective view of the bone anchor delivery device shown in FIG. 13A with a bone anchor implant in place for delivery to a bone with a bone anchor and a suture through an orifice in the bone anchor.

FIG. 13E is a perspective view of the bone anchor delivery device shown in FIG. 13D without a bone anchor implant and showing a rod used to force the bone anchor implant out of the delivery device.

FIG. 13F is a perspective view of a bone anchor delivery device delivering a bone anchor implant to a hole in a bone.

FIG. 13G is a side view of a bone anchor implant with a bone anchor and suture.

FIG. 13H is a side view of a bone anchor implant shown in FIG. 13G.

FIG. 14A is a side view of a bone anchor delivery device with a bone anchor precursor with four arms in place for delivery to a bone.

FIG. 14B is a perspective view of the bone anchor delivery device shown in FIG. 14A.

FIG. 14C is a perspective view of the bone anchor delivery device shown in FIG. 14A and FIG. 14B without a bone anchor.

FIG. 14D is a perspective view of the bone anchor delivery device shown in FIG. 14A with a bone anchor implant in place for delivery to a bone with a bone anchor and a suture through an orifice in the bone anchor.

FIG. 14E is a perspective view of the bone anchor delivery device shown in FIG. 14D without a bone anchor implant and showing a rod used to force the bone anchor implant out of the delivery device.

FIG. 14F is a perspective view of the bone anchor delivery device shown in FIG. 14D delivering a bone anchor implant to a hole in a bone.

FIG. 14G is a side view of the bone anchor implant shown in FIG. 14F with a bone anchor and suture.

FIG. 14H is a side view of a bone anchor implant shown in FIG. 14G.

FIG. 15 is a perspective view of a bone anchor implant with a bone anchor, suture, and cinch bar the suture which has been delivered into a hole in a bone and which has four heat set anchor arms which have not yet reverted to the heat set configuration.

FIG. 16 shows a shape memory alloy ribbon produced by rolling a FIG. 13A is a side view of a bone anchor delivery device with a bone anchor in place for delivery to a bone.

FIG. 17 shows a precursor for a bone anchor implant component produced by electrical discharge machining cutting into a shape memory alloy sheet, including cross-sections of the edges produced by the cutting.

### DETAILED DESCRIPTION

Described herein are bone implant systems and methods of using them that may be especially useful for repairing soft tissue injuries, such as rotator cuff injuries or torn ligaments, in a patient. The bone implant systems and methods described herein include bone anchors, precursors for bone anchors, methods for preparing bone anchors, methods for preparing bone anchor precursors, assemblies for inserting bone anchors, methods for preparing such assemblies, methods for using such assemblies to insert bone anchors, components for use in such assemblies, and substrates having the bone anchors secured therein.

Implants

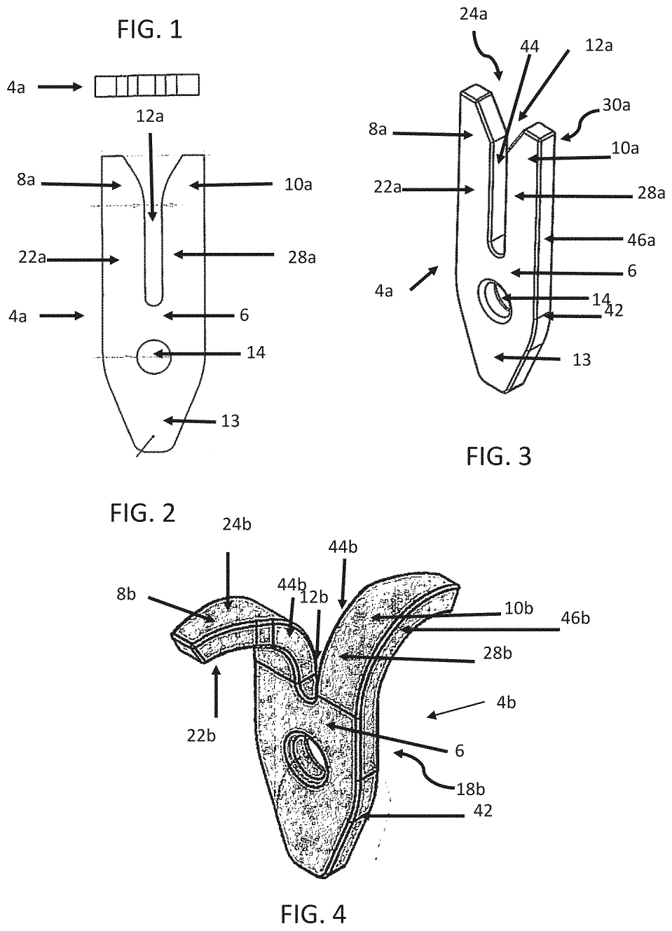

FIG. 1, FIG. 2, and FIG. 3 are top, side and perspective views, respectively, of a bone anchor precursor that may be useful for attaching soft tissue to a bone. FIG. 2 and FIG. 3 show a bone anchor precursor 4a with a body portion 6 connected to two anchor arms, a first anchor arm 8a and a second anchor arm 10a. The first anchor arm 8a and the second anchor arm 10a are separated by a first slot 12a and are on the opposite end from distal end 13 separated by the body portion 6. (Although the terms “proximal” and “distal” are used herein for ease of description to describe bone implants, the implants may have the opposite or another configuration). The first anchor arm 8a and the second anchor arm 10a extend away from the body portion 6. The bone anchor precursor 4a also has an orifice 14 configured to receive a suture. FIG. 2 and FIG. 3 show the body portion 6 has a planar body front surface 16 on the front side of the body portion and a planar body back surface 18 on the back side of the body portion which is parallel to the body front surface 16. In some variations, some or all of the body front surface 16 and the body back surface 18 may not be parallel (e.g., they may be non-parallel). For example, the body portion 6 may be wedge shaped and the body front surface 16 and the body back surface 18 may be in part or in whole oblique to one another. As best illustrated in FIG. 3, the first anchor arm 8a has a first arm front surface 22a on the front side of the first anchor arm 8a and a first arm back surface 24a on the back side of the first anchor arm 8a which is parallel to the first arm front surface 22a. In some variations, some or all of the first arm front surface 22a and the first arm back surface 24a may not be parallel (e.g., they may be non-parallel). For example, the arm may be wedge shaped and the first arm front surface 22a and the first arm back surface 24a may be in part or in whole oblique to one another. Similar to as just described for the first anchor arm 8a, the second anchor arm 10a has a second arm front surface 28a on the front side of the second anchor arm 10a and a second arm back surface 30a on the back side of the second anchor arm 10a which is parallel to the second arm front surface 28a. In some variations, some or all of the second arm front surface 28a and the second arm back surface 30a may not be parallel (e.g., they may be non-parallel). For example, the arm may be wedge shaped and the second arm front surface 28a and the second arm back surface 30a may be in part or in whole oblique to one another. In some variations, the first arm front surface 22a, the second arm front surface 28a and/or the body front surface 16 are planar and coplanar (and lie in the same plane). In some variations, the first arm back surface 28a, the second arm back surface 30a and/or the body back surface 18 are planar and coplanar (and lie in the same plane). In some variations, the first arm front surface 22a, the second arm front surface 28a and/or the body front surface 16 are coplanar and on a different plane from the first arm back surface 24a, the second arm back surface 30a and/or the body back surface 18 which may be planar and coplanar (and lie in the same plane). FIG. 3 also shows inner arm peripheral surface 44a which is adjacent the slot 44a and on the inner surfaces of the first anchor arm 8a and second anchor arm 10a. The inner arm peripheral surface 44a joins the first arm front surface 24a and the first arm back surface 26a. Inner arm peripheral surface 44a also joins the second arm front surface 28a and the second arm back surface 30a. FIG. 3 also shows peripheral outer arm surface 46a which is remote from the first slot 12a and which joins the first anchor arm front surface 22a and first anchor arm back surface 24a on bone anchor precursor 4a. The peripheral outer arm surface 46a may be continuous with a peripheral body surface 42a. Although shown for the second arm of the bone anchor precursor, the first arm similarly includes a peripheral outer arm surface that may be continuous with a peripheral body surface. FIG. 1 shows a top view of the bone anchor precursor 4a, with the sections illustrating the end of the first arm 8a, the proximal peripheral inner arm surface on first arm 8a, the distal peripheral inner arm surface on first arm 8a, the distal-most part of the distal peripheral inner arm surface between first arm 8a and second arm 10a, the distal peripheral inner arm surface on second arm 10a, the proximal peripheral inner arm surface on second arm 10a, and the end of the second arm 10a.

As discussed in more detail below, the implants described herein may be configured to take two different shapes or more than two different shapes. The implants described herein may include a bone anchor precursor, a bone anchor, a cinch bar, and/or a cinch or suture. The implants may include a shape memory material and may be configured to have a first, original shape and/or a second, deformed shape and to transition between the first shape and the second shape. FIG. 4 shows the implant of FIG. 1-FIG. 3 transitioned from the first shape to the second, deformed shape. FIG. 4 shows a bone anchor 4b with first arm 8b bent and second arm 10b bent. The first arm 8b and the second arm 10b are bent away from the body portion in opposite directions. The first arm 8b is bent towards the first arm front surface 22b and away from the first arm back surface 24b. In contrast, the second arm 10b is bent towards the second arm front surface 28b and away from the second arm back surface 30b. In this example, the first arm front surface 22b is concave (along the long axis of this anchor) and the first arm back surface 24b is convex while the second arm front surface 28b is convex (along the long axis of this anchor) and the second arm back surface 30b is convex. The bending may be regular or may be irregular. The bending may form a smooth curve or the bending may be sharp. A bend in the first anchor arm 8b may be bent different from a bend in the second anchor arm 10b. In some variations, only one of the arms may be bent or both arms may be bent in the same direction (e.g., the front surfaces may both be concave or convex). The second, deformed shape may be the shape created when an anchor arm precursor is subject to heat into a “pre-set” configuration. The second, deformed shape may also be the shape created when a bone anchor is inserted into a bone hole and takes on the “pre-set” configuration (when heated by body heat or another heat source) to engage the bone. FIG. 4 also shows that the body portion 6 and the distal end of the implant 13 do not change shape. In some variations, either or both of these may be subject to shape change similar to as described for the first arm and second arm. The implants described herein, including the bone anchor precursors and the bone anchors may be made from a shape changing material, such as a shape memory alloy. The shape memory material may have properties of shape memory effect and pseudoelasticity. The implants may be made from a nickel-titanium alloy, for example an alloy containing 55-56% by weight nickel and the balance titanium, such as 55.8% titanium. The implants may be made from a nickel-titanium alloy which is 20% to 80% nickel, or 49% to 59% by weight nickel, such as 55-56% by weight nickel and the remainder titanium and/or another material. The implants may be made from 20% to 80% titanium, such as 40% to 55% titanium or 44%-45% titanium and the remainder nickel and/or another material. Other materials may include aluminum, copper, niobium, vanadium, and zinc. An implant as described herein may include materials other than shape memory material, such as a biocompatible polymer such as a biocompatible fiber reinforced polymer or other materials. The implants (e.g., precursors and the bone anchors) can include other components that are made of materials other than a shape memory alloy, or of a shape memory alloy different from that in the monolithic article, providing that such other components are composed of a material that can remain in the human body, and do not have a significant adverse impact on the performance of the bone anchor. The other materials may be integrally formed into an implant or placed (e.g., coated or sprayed) on a surface of the implant. An implant may be monolithic (e.g. formed from a single piece of material and have no adhesive, glue, joints, nails, or screws holding it together) or a portion of an implant may be monolithic (e.g., arms, body, and distal end may be formed from a single piece of material) though the implant may have an outer added material (e.g., a coating or spraying). Also described herein is a method of making a bone anchor including the steps of providing a bone anchor precursor as described herein, bending the arms away from the body portion in opposite directions, and heat setting the bent arms in a new configuration. The components described herein including a shape metal alloy can be modified by suitable treatments at different temperatures.

When the anchor arms are composed of a shape memory alloy, the anchor arms may be heat set into a configuration such that, after the implant (e.g., bone anchor) has been delivered, the anchor arms engage the surface of a hole in a bone into which the bone anchor has been delivered and prevent the implant from being drawn out of the hole, and the implant can be fitted into a delivery device, for the heat set anchor arms to be cooled and folded back to or towards the original configuration (such as those shown in FIG. 2, FIG. 3, FIG. 6, FIG. 7, FIG. 10, or FIG. 11). Because the anchor arms, after they have unfolded, are constrained by the walls of the hole into which the device is delivered, the arms will not usually be able to unfold completely. After the device has been delivered, the arms will continue to press against the walls of the hole, and may partially penetrate the walls of the hole, thus stabilizing the bone anchor in the hole. Pulling the bone anchor upwards by one or more sutures, can increase the engagement between the bone anchor and the hole. The ends of the anchor arms can be shaped or coated to enhance the engagement of the arms with the walls of the hole, such as having a point or taper or having surface roughness or teeth at the ends.

The arms unfold from 0% to 100% of their heat set configuration after being placed in a bone, and in some embodiments unfold at 10%, at least 20%, or at least 30% of full recovery to the heat set configuration. Recovery may be determined as a percent ratio of an angle of the arm (or a portion of the arm) relative to the long axis of the anchor at a recovery time compared with the angle of the arm (or a portion of the arm) relative to the long axis of the anchor heat set configuration. The arms may continue to exert outward forces on the walls of the hole to accommodate relaxation of the bone tissue over time. The arms can be shaped so that they dig deeper into the bone tissue when the bone anchor is pulled upwards by a suture secured to the bone anchor. The width of a precursor (at its broadest point) can, for example, be 1.5 mm-5.0 mm or anything between these values. The height of a precursor can, for example, be 3 mm-12 mm or about 2-4 times, such as about 3 times the width. The end of the body portion and/or the ends of the anchor arms can be shaped to facilitate loading the bone anchor into a delivery device and/or to facilitate the entry of the bone anchor into a hole in a bone, such as having a bevel, a rounded rectangle, or a taper.

FIG. 2-FIG. 3 shows the orifice 14 in the body portion 6 of the bone anchor precursor 4a. Although not all bone anchor precursors or other implants include an orifice, in some embodiments they include an orifice into which a suture or suture loop can be fitted. The orifice may be configured to accept and hold a suture without undue damage to the suture (e.g., large enough and with sufficiently smooth edges). The orifice 14 may be oval, round, rectangular, or square. The orifice 14 may be conveniently made by drilling, particularly if the orifice 14 is round.

Described herein is a bone anchor which comprises a monolithic article which may be composed of a shape memory material and may include a body portion including a first planar body surface, a second planar body surface which is parallel to the first planar body surface, and a peripheral body surface joining the first and second body surfaces, and at least two arms which are separated by a slot and which extend away from the body portion in opposite directions, each of the arms having a first arm surface, a second arm surface which is parallel to the first arm surface, a peripheral inner arm surface which is adjacent the slot and which joins the first and second arm surfaces, and a peripheral outer arm surface which is remote from the slot and which joins the first and second arm surfaces.

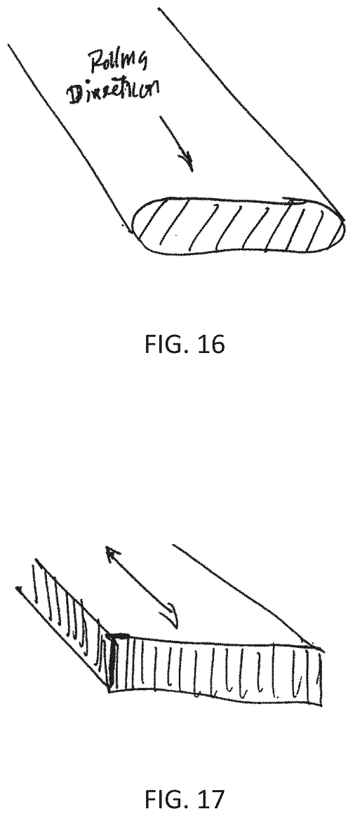

At least part of the peripheral outer arm surface of each of the anchor arms may include a shape, for example a somewhat rounded shape, which is smooth by comparison with a surface produced by cutting into a sheet or ribbon composed of a shape memory alloy. The shape is typical of the edges of a ribbon of a shape memory alloy which has been produced by rolling a rod of the shape memory alloy. The precursor may be produced by modifying a preformed ribbon of a shape memory alloy. FIG. 16 shows a ribbon of a shape memory alloy that is rolled (arrow) to flatten the ribbon into a flat shape with smooth outer edges. In some variations, the width of the ribbon is the same as the desired width of the precursor at its broadest point, which can for example be 1.5-5.0 mm. The edges of the ribbon may provide at least part of the exposed outer surfaces of the anchor arms, thus removing the need and cost involved in creating those edges by one of the available methods for cutting into a shape memory alloy article. Other features of the precursor (for example one or more of the slot between the anchor arms, and/or an orifice for a suture, and/or any desired shaping of the end of the body portion and/or the ends of the anchor arms) can be created using methods such as electrical discharge machining (EDM), and/or a laser, and/or a waterjet, and/or carbide stamping and/or drilling. In some variations, the creation of these features is carried out on the preformed ribbon before cutting the ribbon into discrete parts which are the precursors. Alternatively, one or more of the features can be produced by modification of discrete parts cut from the ribbon. FIG. 17 shows a precursor for a bone anchor implant component produced by electrical discharge machining cutting into a shape memory alloy sheet, including cross-sections of the edges produced by the cutting. The arrow in FIG. 17 shows the rolling direction during fabrication of the underlying ribbon. Alternatively, the precursor may prepared by cutting the desired shape from a sheet of a shape memory alloy. All of the edges in the resulting precursor will then have the square shape that results from conventional methods for cutting into shape memory alloy. Alternatively, the precursor may be produced by a carbide stamping process. The carbide stamping process may be particularly appropriate for precursors of small size.

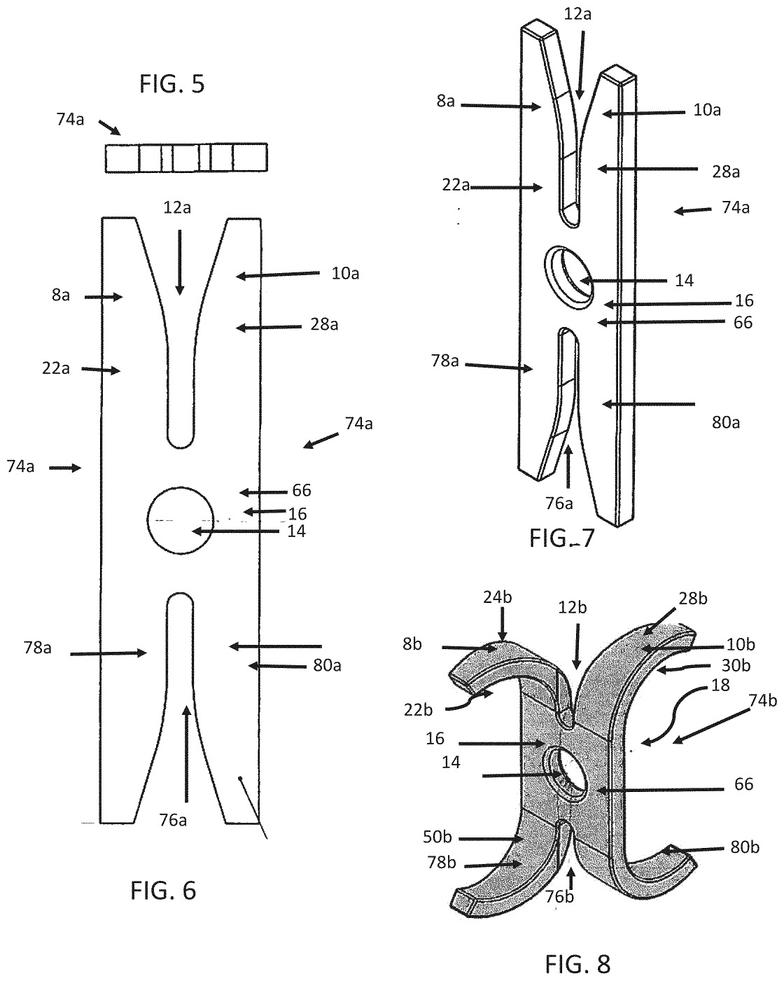

The precursor and the bone anchor comprise at least two anchor arms which extend away from the body portion (e.g., along a central longitudinal axis). There can be one or more additional anchor arms which extend away from the body portion in a direction different from the first two anchor arms, for example two additional anchor arms which are separated by a slot, FIG. 5, FIG. 6, and FIG. 7 show a bone anchor precursor 74a. The bone anchor precursor 74a is similar to the bone anchor precursor 4a shown in FIG. 1, FIG. 2, and FIG. 3 and includes a first arm 8a and a second arm 10a separated by a slot 12a, except that this system includes a second pair of arms, third arm 78a and fourth arm 80a attached to the body portion 66 on the distal end and separated by a second slot 76a. The third arm 78a and fourth arm 80a are similar to the first arm 8a and a second arm 10a and may have any of the attributes described herein regarding the first arm 8a and a second arm 10a. While the third arm 78a and fourth arm 80a are shown substantially as a mirror image of the first arm 8a and a second arm 10a, they do not need to be a mirror image and so may vary such as with anchor arm/slot length, and slot width. FIG. 5 shows a top view of the bone anchor precursor 74a, with the sections illustrating the end of the first arm 8a, the proximal peripheral inner arm surface on first arm 8a, the distal peripheral inner arm surface on first arm 8a, the distal-most part of the distal peripheral inner arm surface between first arm 8a and second arm 10a, the distal peripheral inner arm surface on second arm 10a, the proximal peripheral inner arm surface on second arm 10a, and the end of the second arm 10a. Likewise, the bone anchor 74b shown in FIG. 8 is similar to the bone anchor 4a shown in FIG. 4 and the first arm 8a and a second arm 10a shown in FIG. 5, FIG. 6, and FIG. 7 have transitioned (bent) from the first shape to the second, deformed shape, except that the bone anchor 74b in FIG. 8 includes a second pair of arms, third arm 78a and fourth arm 80a that can be bent similar as to first arm 8a and second arm 10a. For example, third arm 78a and fourth arm 80a can be bent in the same direction or the opposite direction. With the addition of the third arm 78a and fourth arm 80a, the four arms may be bent in any combination (all arms bent towards the “front”; all arms bent towards the “back”; three arms bent towards the front; any two arms bent toward the front; no arms bent toward the “back”). Additionally any of the arms may remain unbent (e.g., in the original configuration). A bone anchor may have a different number of arms (e.g., 3 arms, 4 arms). The additional anchor arms are heat set in a desired configuration in the same way as first two anchor arms.

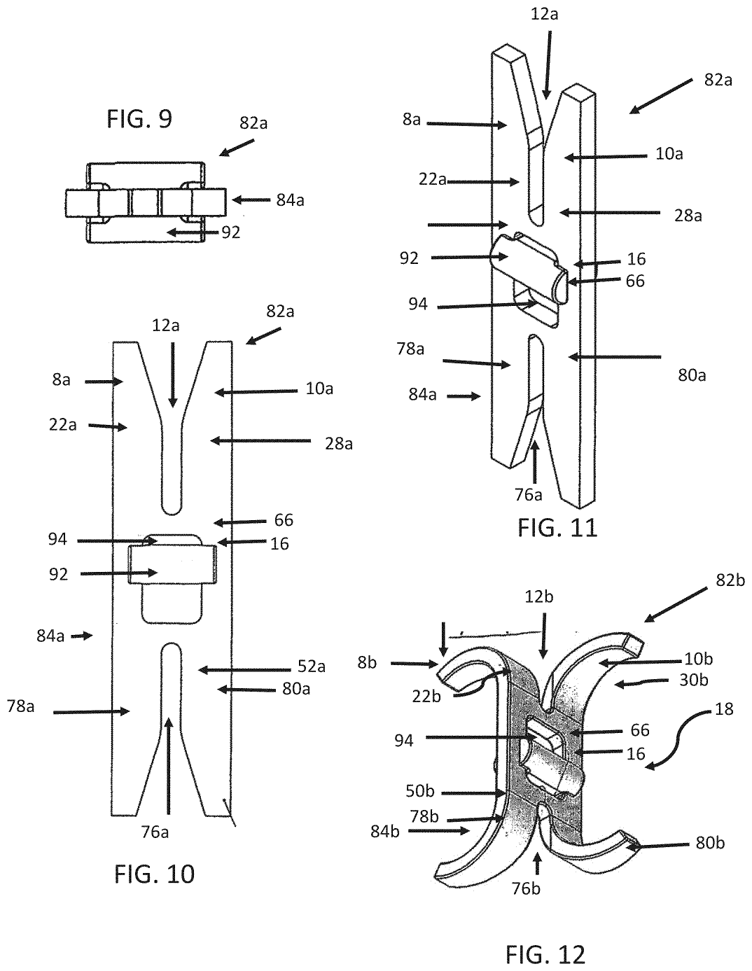

FIG. 9, FIG. 10, and FIG. 11 show another bone anchor precursor 82a. The bone anchor precursor 82a is similar to the bone anchor precursor 74a shown in FIG. 5, FIG. 6, and FIG. 7 and includes two pairs of arms, except that this bone anchor precursor includes a cinch 92 and an orifice 94 configured to accept the cinch bar 92. FIG. 9 shows a top view of the bone anchor precursor 84a which is similar to the top view of the bone anchor precursor 74a shown in FIG. 5 except that this anchor precursor 84a includes a view of cinch bar 92. Likewise, the bone anchor 84b shown in FIG. 12 is similar to the bone anchor 74b shown in FIG. 8 and all four arms, first arm 8a, second arm 10a, third arm 78a, and fourth arm 80a shown in FIG. 9, FIG. 10, and FIG. 11 have transitioned (bent) from a first, original shape to a second, deformed shape, except that the bone anchor 84b includes the cinch 92 that may be especially useful for winding or holding a suture to connect to a soft tissue. The cinch bar 92 may be especially useful for winding or holding a suture to connect the bone anchor to a soft tissue (e.g., a ligament, muscle, or tendon). In one embodiment, a shape memory alloy cinch bar 92 is fitted slidably in the orifice 94 (which may, for example, be generally in the form of a rectangle). This makes it possible for a suture to be passed through the orifice underneath the cinch bar and beyond the end of the bone anchor, forming a loop of two sutures whose lengths can be changed, and then fixed as desired. In order to insert the cinch bar, the orifice (precursor) can be cooled and expanded to enable the cinch bar to be inserted, after which the orifice recovers to the desired shape to hold the cinch bar in place. The body portion of the bone anchor precursor and of the bone anchor can include a slot which communicates with an orifice in the body portion. The slot (i) can be opened, while the bone anchor is in the superelastic state, so that an additional component can be placed in the orifice, and (ii) can then revert to the original shape. The additional component can for example be a suture, a cinch bar, one or more cinch rings, a ring which extends upwards away from the bone anchor arms, or an auxiliary bone anchor. After the additional component has been passed through the slot into the orifice, the slot can be returned to its original configuration, thus trapping the additional component in the orifice.

Delivery Systems

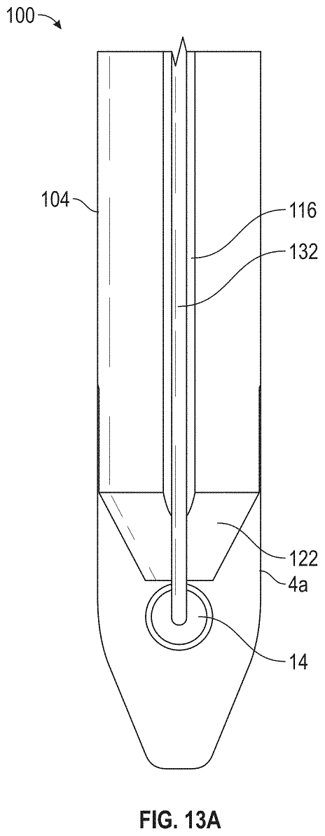

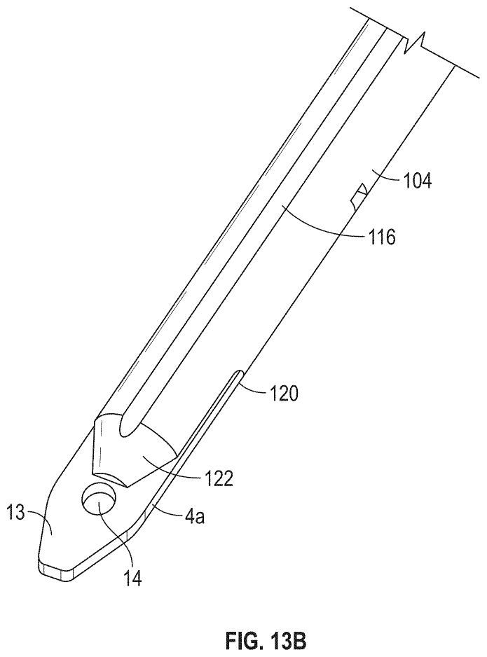

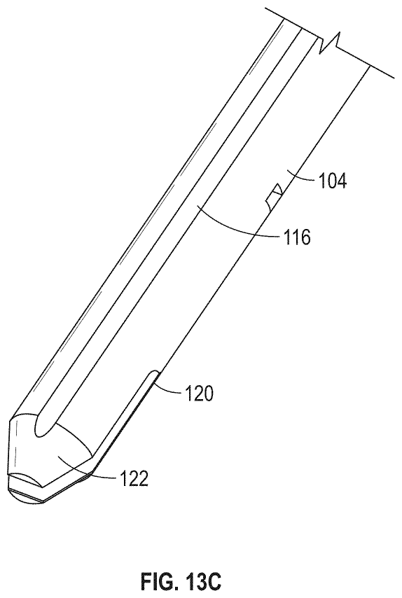

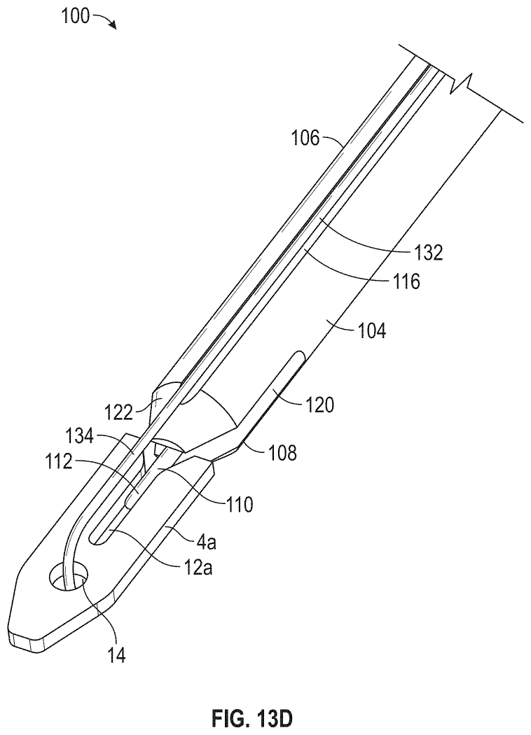

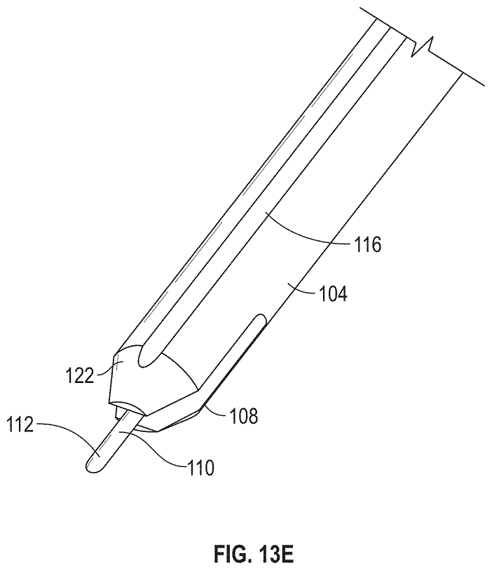

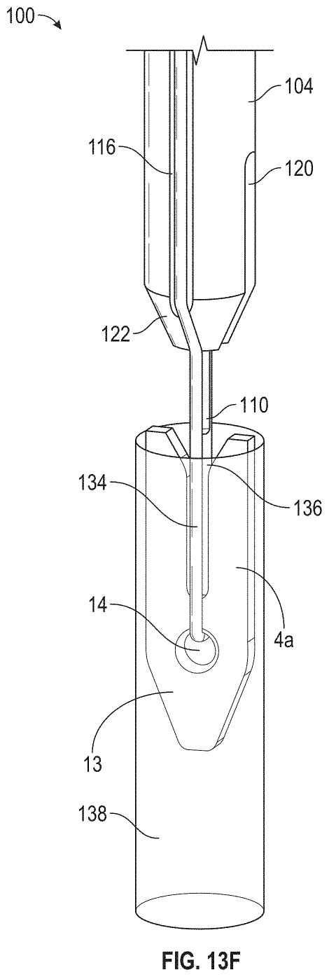

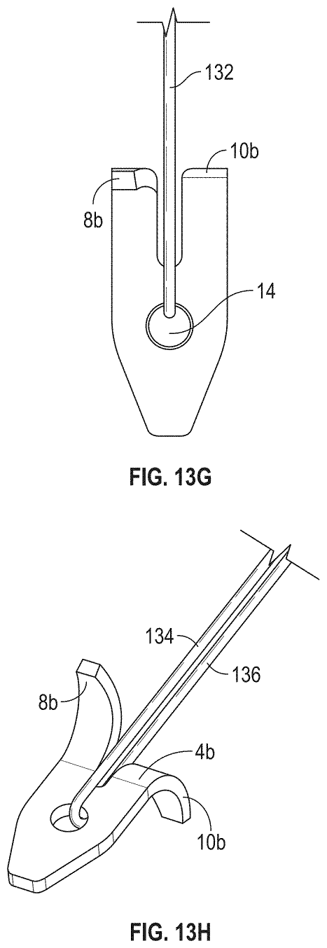

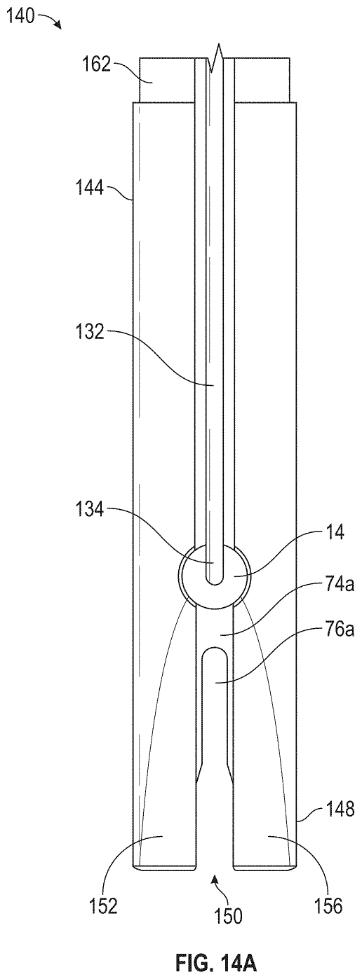

FIG. 13A is a side view and FIG. 13B is a perspective view of the distal end of a bone anchor delivery system 100 with an implant in place for delivery to a hole in a bone. FIG. 13C shows the bone anchor delivery system with the implant (bone anchor precursor 4a with a suture 132) removed to better show the delivery device 104. FIG. 13A and FIG. 13B shows a delivery device 104 holding an implant having a bone anchor precursor 4a with a suture 132 through the orifice 14. A slot 120 on a distal end of the delivery device is configured to accept and hold the bone anchor precursor 4a. The slot 120 (or the entire distal end) may be hollow or open and sized and shaped to accept an implant (e.g., may have a slip fit). The slot 120 have an internal structure feature such as a rough surface, adhesive, or one or more pegs to hold the bone anchor precursor 4a in place prior to insertion of implant into a bone hole. The delivery device 104 also includes a first suture guide 116 configured to accept and/or hold part of a suture for insertion into a bone hold with the bone anchor precursor 4a. FIG. 13A and FIG. 13B also show that the bone anchor precursor 4a is in an original configuration (e.g., the arms are extended). The delivery device 104 includes taper 122. The taper 122 may ease the placement of the delivery device 104 into a bone hole. The taper may be from 15° to 80° (relative to a longitudinal axis of the delivery device 104) such as from 30° to 60°. FIG. 13D is a perspective view of the bone anchor delivery device 104 shown in FIG. 13A showing a pusher 110 for placing a force on the implant and pushing the implant out of the delivery device 104 and placing it into a bone hole and FIG. 13E shows the bone anchor delivery device 104 with the implant removed. The pusher 110 may be shaped to be received by the first slot 12a between the first arm and second arm on the bone anchor precursor 4a. The pusher 110 may engage the peripheral inner arm surface of the bone anchor precursor 4a anywhere along its length. The distal end 112 of pusher 110 may engage the distal-most part of the first slot 12a. The pusher may have a cylindrical shape or a flattened shape. The pusher may have a grip such as a lip or an edge to hold the anchor precursor 4a in place on the pusher 110. FIG. 13D also shows suture 132 looped through orifice 14 for delivery with the anchor precursor 4a into the bone hole. FIG. 13F is a perspective view of the bone anchor delivery system 100 delivering a bone anchor implant to a hole 138 in a bone. The hole in the bone may be made with a drill or spike or may be pre-existing. The pusher 110 has pushed the anchor precursor 4a out of the delivery device 104 and into the hole 138 in the bone. Suture 134 extends from orifice 14 proximally for attaching to e.g., a soft tissue at the proximal side of the bone. FIG. 13G is a side view of a bone anchor implant with a bone anchor and suture as it would appear in a bone hole. FIG. 13H is a side view of a bone anchor implant shown in FIG. 13G. After being placed in the bone hole such as by delivery device 104, the anchor precursor 4a (e.g., the arms) may be heated and the first arm 8a and second arm 10a extend away from the body portion in opposite directions and the extended first arm 8b and extended second arm 10b of the bone anchor 4b can grip the wall of the bone hole. The anchor precursor 4a (e.g., the arms) may be heated from the heat of the body of the patient being treated or by added heat (e.g., from a medical professional applying heat to the anchor precursor 4a) to move the arms from the original configuration to the deformed configuration. The first strand 134 and second strand 136 of suture 132 extend proximally and can be attached to or tightened onto a soft tissue such as a ligament, muscle, or tendon. In some embodiments, no heat is applied to anchor precursor 4a once implanted in the bone but its arms extend in opposite directions toward their heat set orientation through spring force once released from delivery device 104. In some variations, the end of the delivery device can be hollow and can have a circular cross-section. The diameter of the circular cross-section can be for example 1-6 mm, e.g. 1.5, 2.5, 3.5 or 5.0 mm, depending upon the size of the bone anchor to be placed in it. The hollow end section can comprise windows so that a suture can be passed through the windows and through an orifice in the body portion of the bone anchor.

Described herein is a method of making a delivery device including providing a device including a handle and a hollow end section attached to the handle and which has an exposed entrance, providing a bone anchor according to another aspect of the disclosure, cooling the heat set anchor arms of the bone anchor, folding the cooled anchor arms of the bone anchor towards the body portion, and inserting the folded anchor arms and the body portion and the folded arms of the bone anchor into the hollow end section of the device. The hollow end section of the device can be cooled before the body portion and the folded arms of the bone anchor are inserted into it.

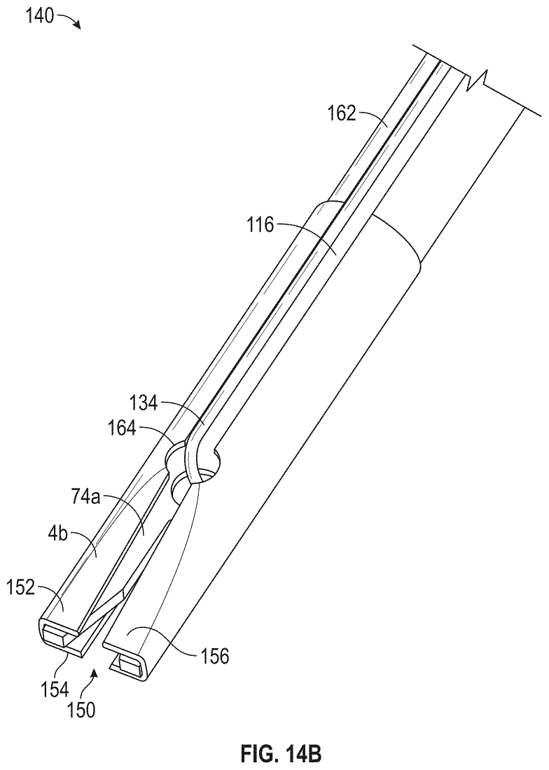

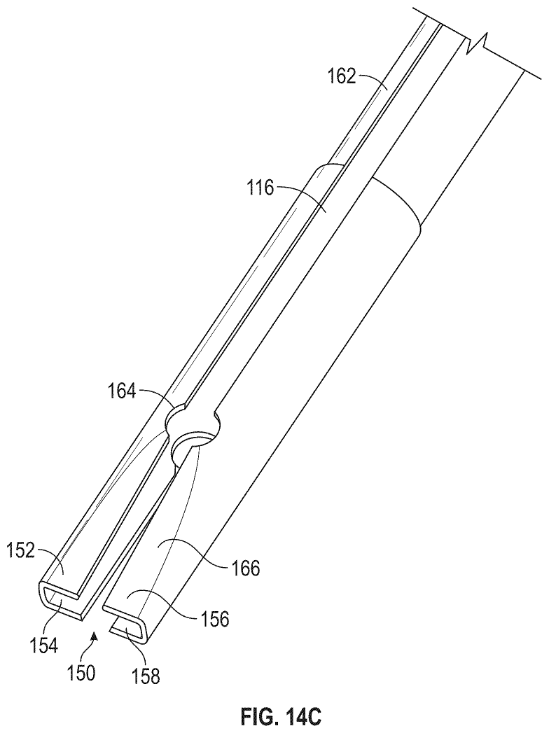

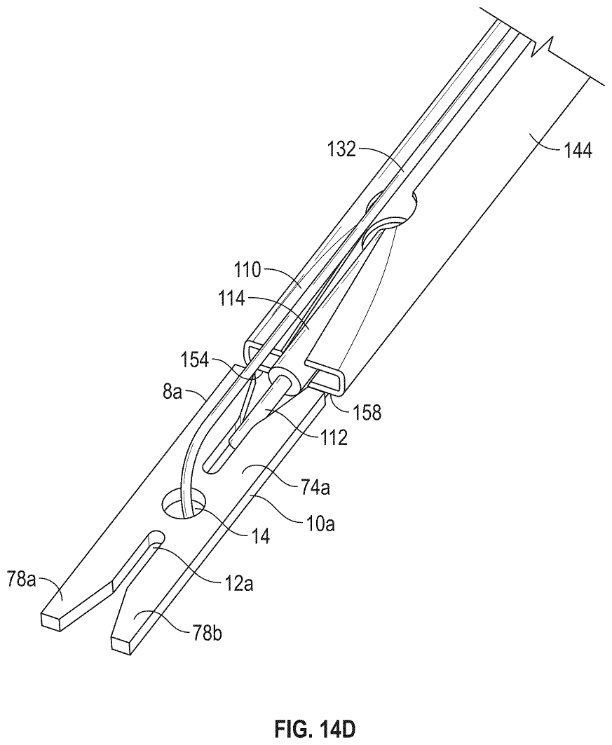

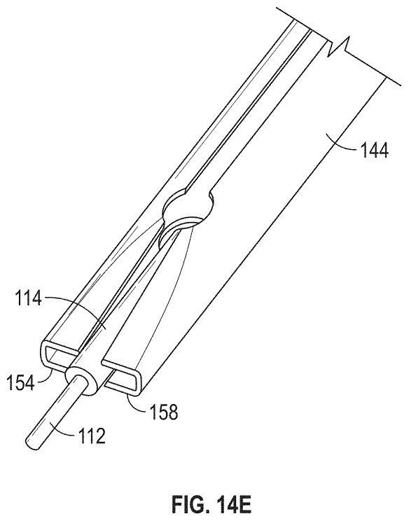

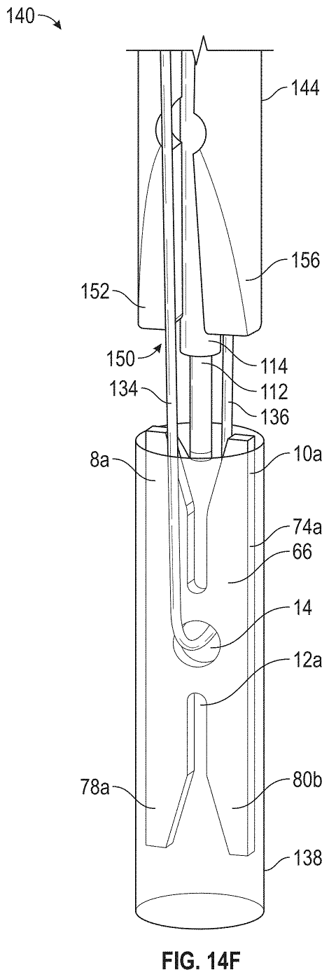

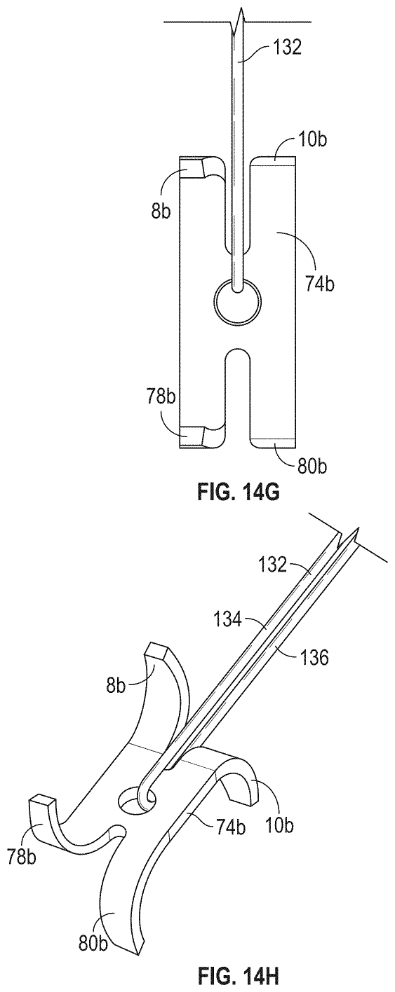

In another variation, a cross-section of the delivery device can be rectangular with rounded corners, corresponding to the cross-section of the bone anchor (with the anchor arms folded) to be placed in the delivery device. Delivery system 140 in FIG. 14A, FIG. 14B, FIG. 14C, and FIG. 14D is similar to the delivery system 100 and includes a delivery device 144 for delivering an implant to a bone hole in a patient, except that the distal end 148 of the delivery device 144 includes a first implant guide 152 with an opening 154 to receive a third arm 78a and a second implant guide 156 with an opening 158 to receive a fourth arm 80a and a space 150 between. FIG. 14A, FIG. 14B, and FIG. 14C also show shaft 162 configured to move through a hollow distal part of delivery device 144. The shaft 162 may be moved by an operator (e.g., a surgeon) to move the pusher 110. Conveniently, but not necessarily, the handle is hollow and within the handle there is a rod that can be pushed through the handle and against the bone anchor, in order to deliver the bone anchor into the hole. The shaft 162 may extend out of the proximal handle (not shown) and the shaft may be moved distally by pushing or twisting a knob on the proximal handle. A shaft similar to shaft 162 may be configured to move through pusher through the hollow distal part of delivery device 104 (see e.g., FIG. 13D and FIG. 13F). The distal end 148 of the delivery system 140 includes a taper 166 configured to fit into a hole in a bone. FIG. 14E shows the delivery device 144 with the implant removed. FIG. 14F shows a delivery device similar to the system 100 inserting a bone anchor precursor into a hole 138 in a bone. In this view both the first strand 134 and the second strand 136 of suture 132 are visible. FIG. 14G and FIG. 14H shows bone anchor 74b in place in a bone (not shown) using the bone anchor deliver device 144. Bone anchor 74b is similar to bone anchor in FIG. 13G and FIG. 13H except that the bone anchor 74b has two additional anchor arms, first anchor arm 78a and second anchor arm that extend away from the body portion 66.

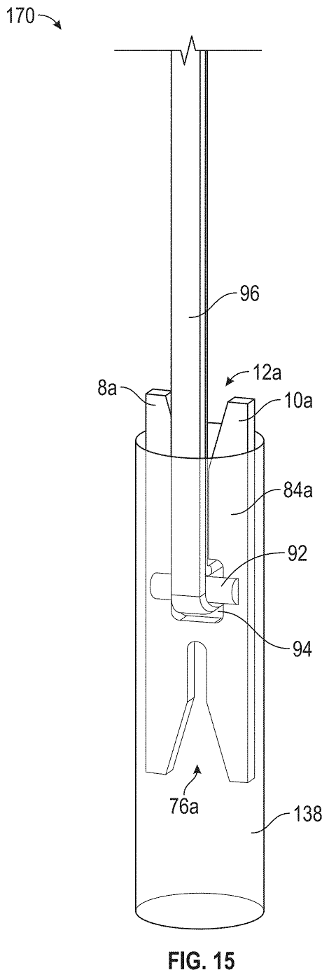

The implant 170 in FIG. 15 is similar to the implant shown in FIG. 14F inserted into a hole 138 in a bone except that the anchor 84a includes a cinch bar 92 for holding a suture ribbon 96.

Also described herein is a device for delivering a bone anchor, the device including a handle, a hollow end section which is attached to the handle and which has an exposed entrance,

a bone anchor within the hollow end section, wherein the anchor arms of the bone anchor are folded from the heat set configuration to or towards their previous configuration, for example so that they are generally coplanar with the body portion. The description of the precursors, of the bone anchors, of the preparation of the bone anchors, of the use of the bone anchors, of the assemblies for inserting bone anchors and of the methods for inserting bone anchors, sometimes refer to the bone anchor as it is to be inserted into a vertical hole in a horizontal substrate (using, for example, terms such as “top”, “bottom”, “upper”, “lower”, “front”, “back”, “horizontal” and “vertical” to refer to different components). This is merely for clarity and convenience, and it should be understood that the disclosed devices can be used to install a bone anchor in any hole in any substrate in any orientation. When the bone anchor has only two anchor arms, it can be installed with the arms being first to enter the hole, with the body portion of the bone anchor above the arms; or it can be installed with the body portion being first to enter the hole, with the anchor arms above the body portion. When the bone anchor has four anchor arms, then two of the anchor arms are first to enter the hole, followed by the body portion, followed by the two remaining anchor arms.

Also described herein is a method of delivering a bone anchor into a hole in a bone, the method including placing a delivery device according to another aspect of the disclosure adjacent the hole in the bone, and delivering the bone anchor into the hole in the bone. If the temperature of the bone is not such that the anchor arms unfold spontaneously, the method includes heating the bone anchor so that the anchor arms unfold. Also described herein is a substrate having a bone anchor according to another aspect of the disclosure which has been inserted into a hole in the substrate and whose anchor arms have reverted to or towards the heat set configuration.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A precursor for a bone anchor having a monolithic article comprising:
a body portion comprising a first planar body surface, a second planar body surface which is parallel to the first planar body surface, and a peripheral body surface joining the first and second body surfaces; and
at least two anchor arms which are separated by a slot and which extend away from the body portion, each of the anchor arms comprising (a) a first planar arm surface which is coplanar with the first planar body surface, (b) a second planar arm surface which is parallel to the first planar arm surface and parallel to the second planar body surface, (c) a peripheral inner arm surface which is adjacent the slot and which joins the first and second arm surfaces, and (d) a peripheral outer arm surface which is remote from the slot and which joins the first and second arm surfaces,
wherein the monolithic article comprises a shape memory material.

2. The precursor of claim 1 wherein the shape memory material comprises a nickel-titanium alloy.

3. The precursor of claim 1 wherein the shape memory material comprises a nickel titanium alloy containing about 55-56% by weight of nickel.

4. The precursor of claim 1 wherein the shape memory material comprises a nickel titanium alloy containing about 44-45% by weight of titanium.

5. The precursor of claim 1 wherein the body portion comprises an orifice configured to pass a suture therethrough.

6. The precursor of claim 4 further comprising a slidable cinch bar in the orifice.

7. The precursor of claim 1 which comprises no more than two anchor arms.

8. The precursor of claim 1 which comprises four anchor arms.

9. A bone anchor having a monolithic article comprising:
a body portion comprising a first planar body surface, a second planar body surface which is parallel to the first planar body surface, and a peripheral body surface joining the first and second body surfaces; and
at least two arms which are separated by a slot and which extend away from the body portion in opposite directions, each of the arms comprising (a) a first arm surface, (b) a second arm surface which is parallel to the first arm surface, (c) a peripheral inner arm surface which is adjacent the slot and which joins the first and second arm surfaces, and (d) a peripheral outer arm surface which is remote from the slot and which joins the first and second arm surfaces
wherein the monolithic article comprises a shape memory material.

10. The bone anchor of claim 9 wherein the shape memory material comprises a nickel-titanium alloy.

11. The precursor of claim 10 wherein the shape memory material comprises a nickel titanium alloy containing about 55-56% by weight of nickel.

12. The bone anchor of claim 9 wherein the shape memory material comprises a nickel titanium alloy containing about 44-45% by weight of titanium.

13. The bone anchor of claim 9 wherein the body portion comprises an orifice configured to pass a suture therethrough.

14. The bone anchor of claim 9 further comprising a slidable cinch bar in the orifice.

15. The bone anchor of claim 1 comprising no more than two anchor arms.

16. The bone anchor of claim 1 comprising four anchor arms.

17. A device for delivering a bone anchor, the device comprising:
(1) a proximal handle; and
(2) a hollow distal section attached to the handle and having an exposed distal end, the exposed distal end configured to receive a bone anchor comprising a body portion having a first planar body surface, a second planar body surface which is parallel to the first planar body surface, and a peripheral body surface joining the first and second body surfaces, and at least two anchor arms which are separated by a slot and which extend away from the body portion in opposite directions, each of the arms comprising (a) a first arm surface, (b) a second arm surface which is parallel to the first arm surface, (c) a peripheral inner arm surface which is adjacent the slot and which joins the first and second arm surfaces, and (d) a peripheral outer arm surface which is remote from the slot and which joins the first and second arm surfaces
wherein the anchor arms are generally coplanar with the body portion.

18. The bone anchor delivery device of claim 17 further comprising an element configured to force the bone anchor out of the exposed distal end after the device has been placed over a hole in a bone.

19. The bone anchor delivery device of claim 17 further comprising a pushrod configured to pass through the handle and/or the hollow distal section and to apply a force on a bone anchor in the exposed distal end.

20. The bone anchor delivery device of claim 18 further comprising a knob or handle on a proximal end of the pushrod wherein pushing on the knob or handle pushes the pushrod through the hollow distal section and applies a force on the bone anchor in the exposed distal end.

21. The bone anchor delivery device of claim 18 further comprising a knob or handle on a proximal end of the pushrod wherein rotating or turning the knob or handle pushes the pushrod through the hollow distal section and applies a force on the bone anchor in the exposed distal end.

22. The bone anchor delivery device of claim 17 wherein a distal end of the pushrod is configured to connect with a proximal end of the bone anchor.

23. The bone anchor delivery device of claim 17 wherein a distal end of the pushrod is configured to fit inside the slot.

24. A method of delivering a bone anchor with a delivery device comprising:
obtaining a bone anchor having a body portion comprising a first planar body surface, a second planar body surface which is parallel to the first planar body surface, and a peripheral body surface joining the first and second body surfaces and at least two shape memory cooled arms which are separated by a slot and which extend away from the body portion in opposite directions, each of the arms comprising (a) a first arm surface, (b) a second arm surface which is parallel to the first arm surface, (c) a peripheral inner arm surface which is adjacent the slot and which joins the first and second arm surfaces, and (d) a peripheral outer arm surface which is remote from the slot and which joins the first and second arm surfaces;
moving the cooled anchor arms of the bone anchor towards a substantially parallel configuration; and
inserting the anchor arms with the substantially parallel configuration into the hollow end section of a bone anchor delivery device comprising a handle and a hollow end section attached to the handle and which has a distal end.

25. A method of making a bone anchor precursor comprising:
modifying a preformed ribbon of a shape memory material to create a bone anchor feature; and
cutting the ribbon into a plurality of discrete parts
wherein the preformed ribbon is the same width as the widest width of the bone anchor.

26. The method of claim 25 wherein the created bone anchor feature comprises at least one of: an anchor arm, an orifice, a slot between two anchor arms, and a slot continuous with the orifice.

27. The method of claim 25 wherein the created bone anchor features include at least two anchor arms, an orifice, and a slot.

28. The method of claim 25 wherein the shape memory material comprises a nickel-titanium alloy.

29. The method of claim 25 wherein the shape memory material comprises a nickel-titanium alloy containing about 55-56% by weight of nickel.

30. The method of claim 25 wherein the shape memory material comprises a nickel-titanium alloy containing about 44-45% by weight of titanium.

31. The method of claim 25 wherein each of the plurality of discrete parts comprises a bone anchor precursor.
